(19) United States
(12) Patent Application Publication
(10) Pub. No.: US 2008/0153087 A1
(43) Pub. Date: Jun. 26, 2008

(54) Method and Apparatus For Orienting Aspherical Cells

(75) Inventor: Andrew Frontin-Rollet, Hamilton (NZ)

Correspondence Address: BROOKS KUSHMAN P.C., 1000 TOWN CENTER, TWENTY-SECOND FLOOR, SOUTHFIELD, MI, 48075, US

(73) Assignee: SELECT XY LIMITED, Hamilton (NZ)

(21) Appl. No.: 10/597,769

(22) PCT Filed: Jan. 19, 2005
(86) PCT No.: PCT/NZ05/00002

§ 371 Date: Aug. 7, 2006

(30) Foreign Application Priority Data
Feb. 5, 2004 (NZ) 530972

(51) Int. Cl. C12Q 1/68; C12M 1/34; C12N 5/00

(52) U.S. Cl. 435/6; 435/287.2; 435/325

(57) ABSTRACT

A method for the orientation of a sperm cell to determine cell differences due to size, mass, or density is used to distinguish X chromosome-bearing sperm cells from Y chromosome-bearing sperm cells and therefore have use in in-vitro and in-vivo fertilization procedures. The orientation of individual sperm cells is determined by measuring non-fluorescent light. The method uses one detector to measure the magnitude of fluorescence (for DNA (sex) measurement from the flat surface of the spermatozoon), and a second detector to measure the magnitude of refracted non-fluorescent light derived from a separate light source. The separate light source is derived from part of a phase contrast or Dark field optical system to provide orientation data. Importantly, all excitation and fluorescent light is excluded from the second detection system by band-pass optical filters thereby providing for a cleaner signal from the concave edge (no fluorescence signal from the flat surfaces of the spermatozoon).

### FIELD OF INVENTION

The present invention generally relates to a method and apparatus for orienting desired cells, or parts of cells, preferably, desired sperm cells and, more particularly, the invention relates to a method and apparatus for orienting, selecting and retaining viable desired sperm cells.

### BACKGROUND OF THE INVENTION

There has been a long felt need for a reliable, qualitative, quantitative and cost-effective method for selecting sperm, which may be used to produce animals of a desired sex

In particular, in the livestock industry farmers or breeders require cows, pigs, sheep, goats, deer, buffalo, horses, etc which are of a preferred sex. For example, bulls are of limited use to a dairy farmer, whereas pig farmers have long been aware that female pigs grow at a faster rate than their male counterparts.

Similarly, cattle and sheep farmers understand only too well that the males of these species produce meat at a faster rate than females.

In mammals the egg carries only the X chromosome whereas the sperm carries either an X or a Y chromosome. The sex of progeny is therefore determined by the sperm cell. When a sperm and an egg are combined and the sperm carries the X chromosome the offspring is female (XX). However, if the sperm carries the Y chromosome, once combined with the X chromosome carried by the female the resultant offspring will be male (XY).

In sperm there is a known difference in DNA content between the X (larger) and the Y (smaller) sperm of for example 3.4% in pigs, 3.9% in cattle and 4.2% in sheep. This measurable difference can be used to determine the sex of the sperm, that is, if it is an X chromosome (female) or if it is a Y chromosome (male) bearing sperm.

The prior art discusses and provides for methods for sorting mammalian sperm into X and Y populations. However, the only reliable methods that maintain sperm viability post-analysis describe the measurement of the DNA mass of individual sperm. These methods essentially use a modified flow cytometer utilising fluorescence measurement to detect what are essentially small differences between the X and Y sperm, wherein the sperm pass single file through a system that measures the DNA content of individual sperm.

Some techniques have been expanded to use a bevelled sample injection tip and a second fluorescence detector in a forward position. This second fluorescence detector is adapted to determine the orientation of flat oval shaped sperm heads with respect to the first detector as they pass through the system.

In both cases it is the magnitude of fluorescence that is being measured. This requires two separate fluorescence detectors, or at the very least two discrete fluorescence readings.

Further adaptations allow for those unwanted sperm to be gated and pass through as waste and discarded.

The prior art therefore describes a flow cytometric system, which requires two separate measurements of the magnitude of fluorescence of the sperm cell, one to determine the sex of the sperm, the other to determine the sperm's orientation. Those skilled in the art would recognise that due to the morphology of sperm cells (flat ovoid shape) and extremely high refractive indices, it is not possible to accurately measure the DNA content of sperm unless said sperm are correctly oriented to the DNA fluorescence detector.

The prior art methods have proven to be expensive—and do not always provide for routine efficiencies much in excess of 80%, although 95% efficiencies have been reported. Furthermore, previously used methods can sometimes overload the photomultiplier tube resulting in a relatively high background noise to signal ratio and an unacceptably high number of incorrectly sexed sperm.

Johnson and Pinkel teach in Cytometry 7: 268-273 (1986), of the provision of two fluorescence detectors, one at 90 degrees and a second at 0 degrees. These detectors simultaneously collect fluorescence signals from the edge and flat side of the sperm nucleus. The fluorescent detector at 90 degrees is used to determine the orientation of an individual spermatozoon orthogonal to a second fluorescence detector, which measures the magnitude of fluorescence and hence total DNA content (and thereby sex) of the spermatozoon.

The prior art disclosed by Lawrence Johnson in U.S. Pat. No. 5,135,759, and assigned to XY Inc., teaches of a method, which measures the magnitude of fluorescence from both detectors to provide relevant data. That is, fluorescence is used to determine both orientation and the DNA content (sex) of any given sperm cell. This Johnson patent does not visit the novel concept of determining orientation using refracted non-fluorescent light emission.

The Johnson method/apparatus is based solely around a modified flow cytometer. The flow cytometer is a commonly used laboratory instrument for the analysis of individual cells and separates the cells into three populations. Essentially the flow cytometer injects cells into a sheath fluid system that teases cells out into single file and orients them within an optical/focal plane. Dependent upon internal geometry, the nozzle may also orient cells radially within the sheath fluid flow.

The cells are then ejected under pressure from the nozzle in a stream of droplets, each droplet ideally containing a single spermatozoon (some droplets contain multiple spermatozoa and some none). Individual spermatozoa are typically optically analysed within the droplets and by means of applying a positive, negative or zero charge to individual droplets, according to analysis, and then passing said droplets between electrically charged deflection plates, sorting into separate populations may be accomplished. Without going into detail the process can be problematic, particularly at high speed. Nevertheless XY Inc. claim sort purities of 90-98% dependent on processing speeds, i.e. the higher the speed the lower the accuracy of sorting.

A significant disadvantage of the Johnson/XY, Inc. process is the viability of the sorted spermatozoa. Droplets exit the nozzle at high speed and dependent upon sorting speeds, droplet velocity may reach speeds of 20 metres per second resulting in huge stresses upon the spermatozoa impacting upon fluids in, or the walls of, the collection vessel. Streaming of tiny droplets into air also exposes spermatozoa to oxidative stress and it is thought that such stresses may affect sperm viability and result in relatively lower yields of viable sperm.

Various nozzle systems are disclosed in U.S. Pat. No. 6,263,745, U.S. Pat. No. 6,357,307 & U.S. Pat. No. 6,604,435. These documents form differing aspects of the same invention. They all relate to an improved nozzle system for a flow cytometer and generally describe a means to accelerate the delivery of sperms cells, hopefully at the correct orientation, to be sorted and analysed. U.S. Pat. No. 5,985,216. This document describes a tapered sorting nozzle. It is reported to be able to both orient and allow for sorting of desired and viable sperms types from a sample. None of the above documents disclose the novel aspects of the present invention.

WO 98/34094 teaches of an epillumination system adapted to a flow cytometer that does not require sperms to be aligned or oriented. In effect it organises and directs the collection of fluorescent light from an illuminated sample stream in a flow cytometer by using a paraboloid or ellipsoid shaped collector. The '094 method gives comparatively slow passage flows and may compromise cell viability.

WO 01/85913 describes a method of analysing the DNA volume of X and Y carrying sperm. The document discusses the use of electromagnetic radiation (or simply light which is electromagnetic radiation) and modified differential interference contrast optics to measure a sex differentiation characteristic such as volume of sperm cell heads. The electromagnetic radiation can be a laser, microwave or UV light. The thrust of the '913 document attacks the problem of orientation, distorted readings and background signals caused by fluorescence measurement. The document states that this “can allow small differences in photoemissive light to be differentiated even when total light emitted from each photoemissive event is high, or even when there are a high number of bright serial events per second”. The '913 patent measures minute changes in phase shift, ie the difference between the waveform characteristics of light prior to and after penetration of the sperm. The document teaches of the use of complicated, modified interference optics and polarised light to determine sample orientation. The use of phase contrast or Dark field optics to measure refracted non-fluorescent light to determine sample orientation is not contemplated.

There is a need therefore for a simple and effective method and apparatus, which enables individual cells to be sorted accurately and quickly from a population of cells and wherein the cells remain viable.

### OBJECT OF THE INVENTION

It is an object of the present invention to provide an improved method and/or apparatus for selecting desired cells, or parts of cells, or is one which will obviate or minimise the foregoing disadvantages or will at least provide the public with a useful choice.

### STATEMENT OF THE INVENTION

Accordingly, a first aspect of the invention provides for a method of determining the orientation of a cell in a process wherein said orientation is used to allow for the determination of cell differences due to size, mass, volume or density and whereby the orientation of the cell is determined by measuring non-fluorescent light.

Preferably, the orientation is determined by measuring light using a band pass filter to exclude all light other than from a phase contrast optical system or a system utilising Dark field optical techniques.

Preferably, the cell is an aspherical cell.

Preferably, the cell is a sperm cell.

Preferably, the method for determining the orientation of the cell does not require the cell to be encapsulated within a droplet

Preferably, the method for determining the orientation of the cell is used in tandem with a method for measuring the DNA content of the cell.

Preferably, the method for determining the orientation of the cell is used simultaneously with a method for measuring the DNA content of the cell.

Preferably, the method for determining the orientation of the cell is used in a method for selecting sperm of a desired chromosome complement.

Preferably, the method for determining the orientation of the cell is further used in a method for differentiating X chromosome bearing sperm from Y chromosome bearing sperm and/or selecting a population of cells having a desired sex.

Accordingly, a second aspect of the invention provides for a method of selecting a desired cell, or parts of a cell, the method having the following steps:
(i) passing suitably maintained cells from a sample of cells of interest into a testing zone,
(ii) exposing said cell sample of interest to a first light source having a first wavelength,
(iii) exposing said cell sample of interest to a second different light source of a second different wavelength,
(iv) collecting light energy emitted at (ii) and (iii) above,
(v) analysing the light collected at (iv) to determine whether the desired predetermined parameters are met,
(vi) selecting those cells, or parts of cells, which meet said desired parameters,
(vii) collecting the selected cells in a suitable viability maintenance medium, and/or
(viii) eliminating those unwanted cells, or parts of cells, as waste.

Preferably, the cells are sperm cells.

Preferably, the sperm cells are stained with a suitable DNA-specific binding fluorochrome.

Preferably, the first light source is of a suitable wavelength(s) to excite fluorescence in said DNA specific binding fluorochrome(s).

Preferably, the first light source develops one or more wavelengths of emitted fluorescent light to enable analysis of the DNA content of a sperm cell.

Preferably, the fluorochrome is selected from SYBR green I, SYBR green II, SYBR gold, and Bisbenzimide H33342

Preferably, the second light source is used to determine the orientation of the cell.

Preferably, the second different light source uses a light source derived from a phase contrast optical system or one using Dark field optical techniques.

Preferably, the cell is simultaneously exposed to said first light energy and second different light energy.

Preferably, the cell is passed through an orientation device wherein the orientation of the cell is hydrodynamically oriented to achieve a uniform radial geometry with respect to the detector(s)

Preferably, the testing zone is a rectangular receiving area adapted to maintain the orientation of single cells, most preferably sperm cells during analysis.

Preferably, the cells to be tested are delivered to the rectangular testing zone at a flow rate, sufficient to maintain and retain cell viability, preferably at above 1,000 cells per second, and most preferably between 1,000 to 100,000 cells per second.

Preferably, the cells to be tested are delivered to the rectangular testing zone at a flow rate of from 5,000 to 40,000 cells per second.

A third aspect of the invention provides for an apparatus for selecting a desired cell, or parts of a cell, the apparatus comprising:
(i) a means for passing suitably maintained cells from a sample of cells of interest into a testing zone,
(ii) a means of exposing said cell sample of interest to a first light source having a first wavelength,
(iii) a means of exposing said cell sample of interest to a second different light source having a different wavelength,
(iv) separate means for collecting and, if necessary, amplifying light emitted by said sample at (ii) and (iii)
(v) a means for analysing the data collected by separate means (iv) to determine whether desired predetermined parameters are met,
(vi) a means for selecting, collecting and maintaining cells in viable condition meeting said desired predetermined parameters, and/or
(vii) a means for eliminating, those unwanted cells, or parts of cells, as waste.

Preferably, said first light source is a source of electromagnetic radiation, such as a laser.

Preferably, said first light source is adapted to allow for the analysis of the DNA content of a cell.

Preferably, said second light source is derived from a phase contrast optical system or a system utilising Dark field optical techniques.

Preferably, said second light source is adapted to determine the orientation of a cell.

Preferably, said means for collecting light emitted from said sample after exposure to said first light source comprises one or more microscope objective(s), or similar.

Preferably, said means for collecting light emitted by said sample after exposure to said second light source is an optical detection system adapted to collect light energy of a non-fluorescent wavelength.

Preferably, said analysis and identification means is a multi-channel analyser or computer programmed with suitably developed computer software.

A fourth aspect of the invention provides for a delivery device for delivering in a laminar flow suitably maintained sperm cells from a sample injection tube via a hydrodynamic radially orienting nozzle and thereafter to a testing zone, the delivery device comprising:

an elongated tube having a first end portion and a second end portion,
the first end portion comprising a nozzle,
the second end portion comprising a pre-collection or deceleration zone, and wherein,
the first and second end portions are spaced apart either side of a substantially rectangular cross-sectioned testing zone and wherein,
said first end portion comprising said nozzle has a first end and a second end, said first end adapted
to communicate with a sample injection tube to receive said sample and said second end being
contiguous with said testing zone, the nozzle being of a size and shape sufficient to maintain said
sperm cells in a laminar flow at a hydrodynamic radial orientation, and
said second end portion comprising said pre-collection or deceleration zone is configured to convey
sperm cells to a collection means such that said cells after exiting the testing zone are maintained in a viable condition suitable for use in an in-vitro or in-vivo fertilisation procedure.

Preferably, the pre-collection or deceleration zone is flared outwards from the substantially rectangular cross-sectioned testing zone.

Preferably, in use, as the cells pass from the injection tube and into the delivery device the orientation nozzle orients and maintains individual cells into a position which allows for each individual cell to pass through a first light source having a first wavelength and light emitted by said cell to be detected and analysed for DNA mass, and which simultaneously allows for said cell to pass through a second different light source having a second different wavelength to be detected and analysed for correct orientation.

Preferably, individual cells are exposed to said first and second light sources simultaneously.

A fifth aspect of the invention further provides for a method of selecting a desired sperm cell, or part of a sperm cell, the method having the following steps:
(i) staining intact, viable sperm collected from a male mammal with a suitable fluorescent dye, such that the DNA takes up the fluorescent dye uniformly,
(ii) maintaining the stained sperm in a suitable maintenance medium sufficient to maintain the sperm and/or contained DNA within the cell in a viable condition,
(iii) passing the maintenance medium containing the sperm before a suitable excitation light source causing the stained DNA to fluoresce,
(iv) passing the maintenance medium containing the sperm through both a means for measuring the fluorescence of the stained DNA and a means for detecting the orientation of the sperm,
(v) collecting light energy emitted by said sperm cell, converting the light energy into electrical signals and analysing the electrical signals via a multi-channel analyser or suitably programmed CPU,
(vi) selecting those sperm cells, or parts of sperm cells meeting desired predetermined criteria, and
(vii) a means for eliminating those cells, or parts of cells, which fail to meet the desired predetermined criteria.

### SUMMARY OF THE INVENTION

The prior art disclosed by Lawrence Johnson in U.S. Pat. No. 5,135,759, and assigned to XY Inc., teaches of a method, which uses both a 90 degree and 0 degree optical detector to collect and measure the magnitude of fluorescence to determine both orientation and the DNA content (sex) of any given sperm cell.

By contrast, the present invention provides for a novel method, which uses a first detector to measure the magnitude of fluorescence for DNA measurement from the flat surface of the spermatozoon), and a second detector to measure the magnitude of refracted non-fluorescent light derived from a separate light source. The separate light source is derived from part of a phase contrast or Dark field optical system to provide orientation data. Importantly, all excitation and fluorescent light and any unwanted or aberrant light from any other sources is excluded from the second detection system by appropriate band-pass optical filters thereby providing for a cleaner signal from the concave edge (ie any fluorescence signal emitted from the flat surfaces of the spermatozoon is excluded and not measured). The use of phase contrast or Dark field optics to measure said non-fluorescent light achieves a significant lesser loading of the PMT. This reduction in PMT loading therefore allows for higher processing speeds, economies in processing costs and significantly higher sperm viability retention due to shorter individual sample processing time. The Johnson method is speed limited as higher processing speeds can result in an undesirable high background noise to signal ratios created by signal bounce.

Surprisingly, the present inventor has found that a process wherein the orientation of a sperm cell is determined by passing light using optical phase contrast or Dark field techniques through a sperm cell of interest provides for improved efficiencies and increased reliability in the results obtained. In other words orientation of sperm cells—the correct orientation defining whether a result should be accepted for further analysis—can be determined by measuring non-fluorescent light emitted by a sperm cell.

The use of phase contrast optics or Dark field optical techniques as a means to measure refracted non-fluorescent light has never before been considered as a means to determine the orientation of aspherical cells.

The inventor has also found unlike the prior art that there is no need for the cells of interest to be encapsulated or confined within an electrically charged medium during the analysis and collection phase of the process. Previously, once the DNA content of a sperm cell had been determined, the cells were encapsulated in a droplet to which is appended an electric charge, the charge being dependent on a cell's X or Y sex chromosome content. The droplets were then separated based upon the charge they received. The present invention simply selects those cells having a desired sex chromosome based upon predetermined parameters programmed in the analyser. If the criteria are met the cell passes untreated and is retained in a viable condition suitable for either in-vivo or in-vitro fertilisation uses. If the criteria are not met, the cell may be permanently immobilised through permeabilisation of the plasma membrane by heat transference generally, but not necessarily, by exposure to a laser or, partially or completely destroyed by a process of ablative photodecomposition, generally by exposure to a laser.

This invention also teaches the use of a rectangular testing zone located downstream of an orienting nozzle. A cell emerging from the orienting nozzle can be maintained at the correct radial orientation to allow for accurate DNA analysis. The substantially rectangular configuration of the testing zone of the invention has been found to provide for superior accuracy and more reliability in the results obtained. Previous testing processes maintain the cell being measured in a circular cross sectional fluid stream or liquid droplet, which is of an essentially elliptical or circular configuration as the cell emerges from the nozzle. This configuration, although allowing for commercially acceptable cell flow rates of a desired orientation, also allows for inaccuracies due to light being refracted from the curved surfaces of the fluid stream or droplets being measured.

Importantly, this rectangular testing zone provides for four flat surfaces. The improvement results in a significant reduction in unwanted refracted light over systems where curved surfaces are used thus eliminating false readings. As such the provision of four flat surfaces provides for a much-improved reliability over previously disclosed systems.

The present invention therefore comprises at least four novel components, the aspects of which will be outlined later in greater depth. Firstly, the invention uses phase contrast or Dark field optics to determine a desired cells orientation with respect to a DNA measurement detector. Secondly, the invention makes no requirement for the cells of interest to be encapsulated in droplets or otherwise to enable desired cells to be physically separated from those that are not wanted. Thirdly, the use of a substantially rectangular testing zone reduces the effects measurement of unwanted light has on the process. Fourthly, the invention teaches of a laser actuated means for temporarily or permanently immobilising or even destroying unwanted cells.

The above features therefore provide significant, surprising and novel advantages over existing cell selection/sorting processes and in particular over those processes directed to the sexing of sperm cells.

### DETAILED DESCRIPTION OF THE INVENTION

The following examples are illustrative only and, where specific integers are mentioned which have known equivalents, such equivalents are deemed to be incorporated herein as if individually set forth.

The examples describe preferred embodiments only and are non-limiting.

The present application has particular relevance in the selection of sperm cells carrying a desired sex chromosome. The ability to provide for populations having viable X chromosome bearing sperm or Y chromosome bearing sperm at purity of 95% or even 98% or more is now achievable.

### BRIEF DESCRIPTION OF THE DRAWINGS

Various other objects and features and attendant advantages of the invention will become more fully appreciated as the same becomes understood in conjunction with the accompanying drawings, in which like reference characters designate the same or similar parts throughout several views, and wherein

FIG. 1 is a schematic showing the system methodology.

FIG. 2 is a flow chart of the general process.

FIG. 3 illustrates the delivery device.

FIG. 4 illustrates the injection tube, delivery device and collection point relationship.

FIG. 5 shows the cross-sectional relationship of components comprising the apparatus as seen through line ‘A-A’ of FIG. 4.

FIG. 6 provides an overview of the components comprising the apparatus.

### EXAMPLE 1

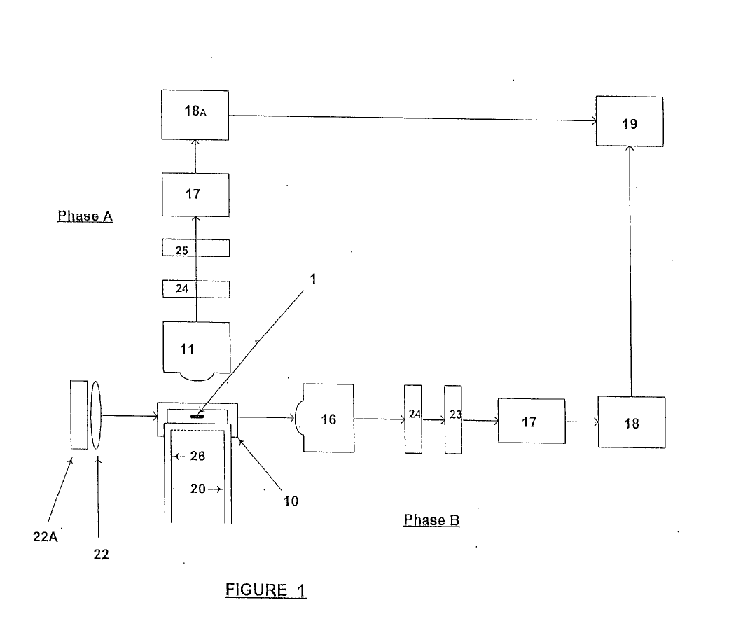

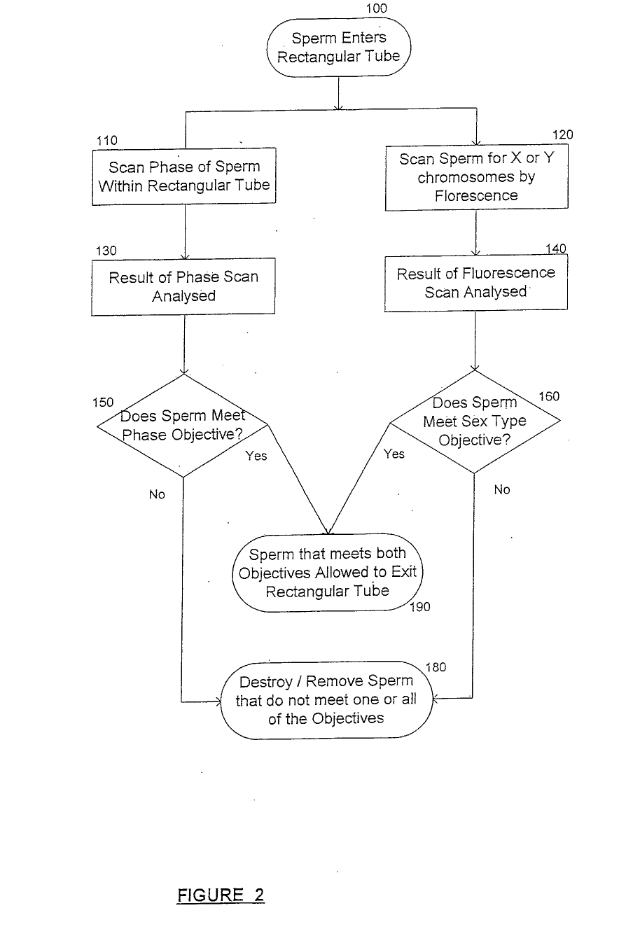

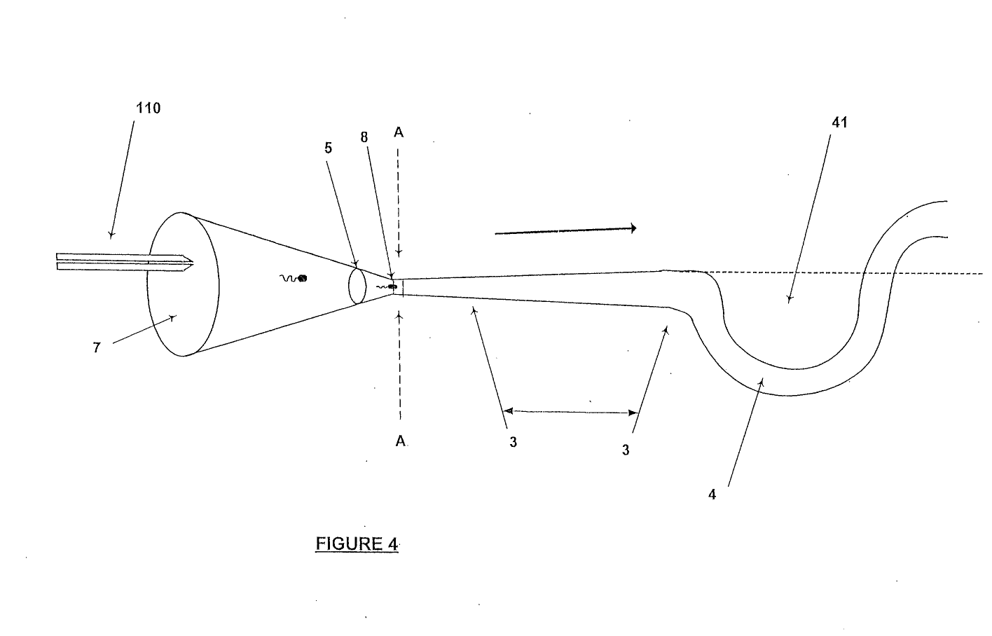

Turning now to FIGS. 1 to 6, the various apparatus used and method steps involved in the process are described in detail.

Live sperm to be differentiated according to their sex characteristic are collected by standard collection techniques and maintained in a suitable medium such as a Tris buffer medium. The DNA within the cells is stained with a non-toxic fluorochrome, preferably SYBR green I, SYBR green II, SYBR gold or Bisbenzimide H 33342. Intact stained sperm are then subjected to a fluorescence excitation energy source provided by an optical fibre or hollow glass fibre (26). The preferred excitation wavelength is about 488-497 mm and is dependent on the particular fluorochome being used. Signals emitted are collected via a fluorescence collection objective (11) or similar, measured by a photo multiplier tube (PMT) (18A) and processed/analysed by a suitably programmed CPU/analyser (19) to determine orientation of individual sperm. If after analysis the sperm cell under investigation meets desired criteria the sperm cell is selected, collected and maintained in an appropriate maintenance fluid—for later use in in-vivo or in-vitro fertilisation procedures.

Those cells that do not meet the predetermined criteria are either permanently immobilised by a process of heat transference (generally but not necessarily by exposure to a laser) or are destroyed by a process of ablative photodecomposition, generally by exposure to a laser (20).

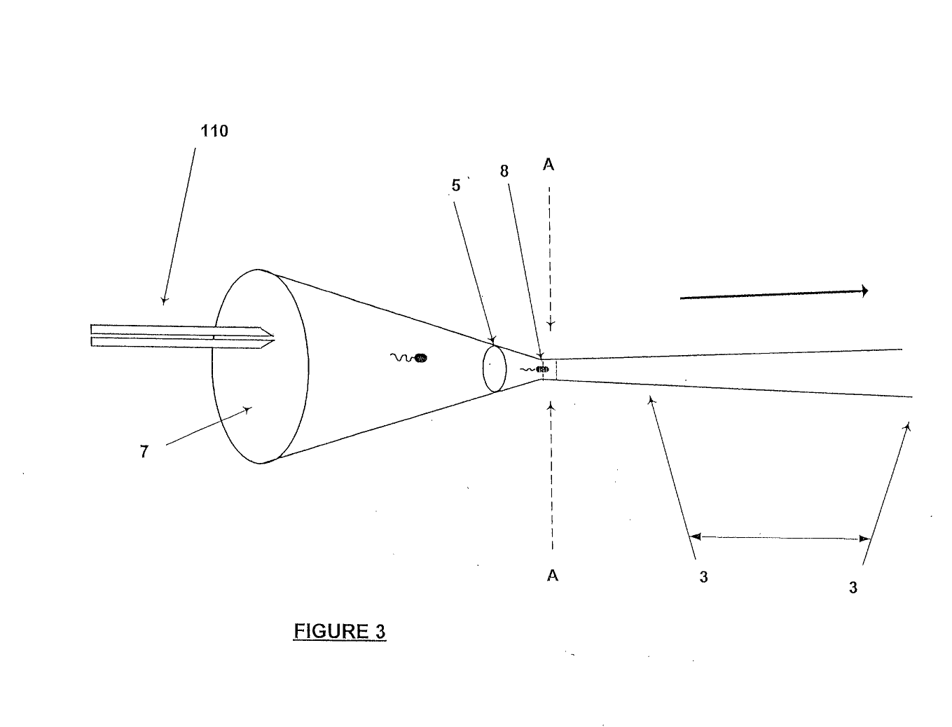

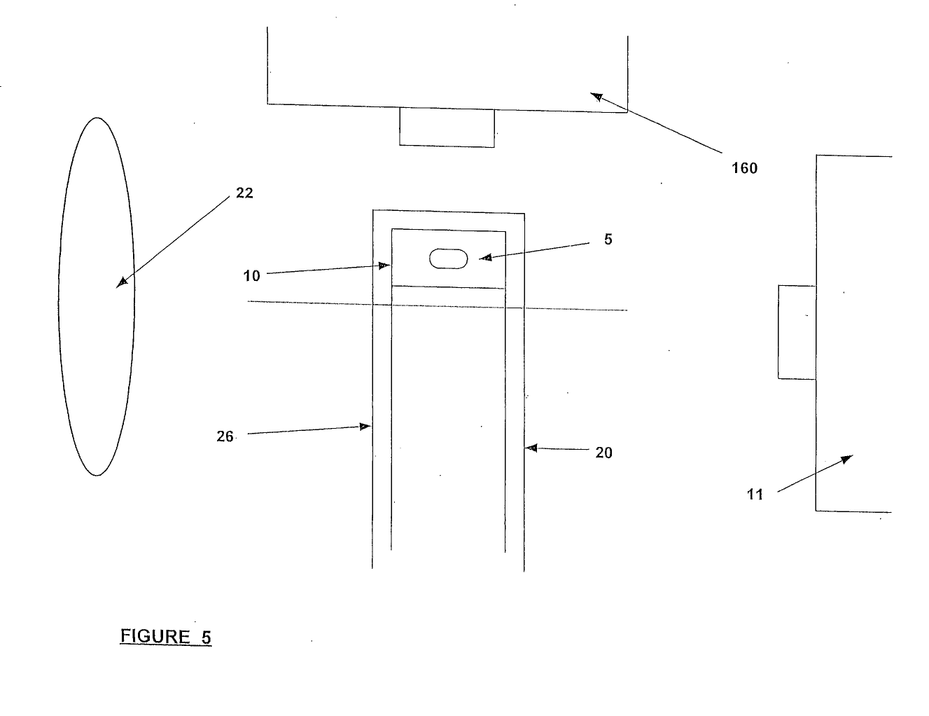

Referring now to FIGS. 1, & 3-6, individual sperm cells are allowed to pass in single file through a nozzle (8) (See FIG. 3) and into a testing zone (10). The testing zone (10) (see FIG. 5) is generally rectangular in shape and is of a dimension, which allows for individual sperm cells to be accommodated and their orientation maintained for DNA analysis. The flow of sperm cells is continuous throughout the process. Processing flow rates of about 10,000 to 35,000 sperm per second are contemplated, although flow rates of between 1,000-100,000 per second are thought to be possible. The flow rate will be such that the sperm remain viable and will depend on system factors. Factors include the pressure at which the system is run, which is likely to be between 30 psi and 70 psi and the intensity/PMT loadings and laser repetition rate of about 3 to 300 KHz.

The DNA analysis and selection of desired sperm comprises two phases. The phases are preferably conducted simultaneously, but not necessarily. There may be occasions when the phases are concomitant, for example when individual sperm queue after analysis before unwanted sperm are immobilised. There may also be instances when sperm leaving the testing zone undergo further tests before being retained or discarded depending on predetermined criteria.

In phase A, an individual sperm cell (1) has previously been stained with a fluorochrome. The fluorochrome binds to the DNA. The amount of fluorochrome that binds to the DNA is dependent on the amount of DNA present. Given that an X chromosome contains more DNA than a Y chromosome, a female sperm (X) will take up a greater measurable amount of fluorochrome than does a male sperm

The more fluorochrome taken up, the more fluorescence is emitted, and the differences between individually fluorescing cells can be measured.

Individual sperm cells pass through the rectangular testing zone (10) and are exposed to a fluorochrome excitation light source (27) delivered via a hollow, rectangular glass fibre (26). The fluorochrome bound to the DNA is excited and fluoresces. The fluorescence is collected through an objective (11), filtered by an appropriate band pass filters (24,25) to filter all non-fluorescent light and collected by a PMT (18A), and forwarded to a suitably programmed CPU/analyser (19) for analysis.

Referring to FIG. 1, Phase B operates simultaneously with Phase A. Here, individual sperm (1) arriving at the rectangular testing zone (10) are subjected to a phase contrast or Dark field optical condenser (22) and whereby refracted non-fluorescent light emitted from the sperm being tested is collected. Bandpass filter (24) are used to ensure that any residual fluorescent light or any other unwanted light occurring in bandwidths (450 nm-550 nm) is excluded. The refracted light is optionally filtered through a further filter (24) to exclude electromagnetic energy emitted from the heat transference or ablative photo decomposition laser, collected by a PMT (18), and transported to a suitably programmed CPU/analyser (19) for analysis. Utilisation of the above phase contrast or Dark field orientation determination method essentially requires that all measurable electromagnetic energy other than that derived from the phase contrast system (16, 22, 22A) be excluded from measurement by the 90 degree PMT system, through the provision of appropriate optical filters (23,24).

Once analysis is completed those cells not meeting predetermined criteria are permanently immobilised by a process of heat transference or destroyed by an ablative photodecomposition device (laser). The laser/immobiliser input (21) is located downstream of the analysis/measurement processing point and is controlled by the CPU/analyser (19).

FIG. 1 is described by the following:
1. cell
10. testing zone (rectangularly configured to provide four substantially flat surfaces)
11. Fluorescence collection objective
16. Phase contrast or Dark field objective
17. Pre-amplifier (optional)
18. PMT
18A. PMT
19. CPU/Analyser
20. Immobilising external triggered Q-switched laser or Ablative external triggered Q-Switched laser
21. Optical fibre or hollow rectangular glass fibre to deliver immobilising or ablative energy
22. Phase contrast or Dark field condensers
22A. Second Light Source for the Phase Contrast or Dark field optical system
23. Band pass filter to exclude wavelengths from 450 nm-550 nm
24. Band pass filter to exclude aberrant unwanted light from 20.
25. Band pass filter to exclude all non-fluorescent wavelengths
26. Optical fibre or Hollow rectangular glass fibre to provide Fluorescent excitation light.
27. Fluorescence excitation light source.

A means comprising a second geometric axial motion system allowing gentle deceleration of desired cells to be collected via a pipette or the like and maintained in a suitable medium for later use is also contemplated. The collection means is located downstream of the delivery device and the testing zone and will act much like a groyne in a river system. See Example 4.

### EXAMPLE 2

The device shown in FIGS. 3 to 6 illustrates the mechanism by which suitably stained and intact sperm are provided for testing as described previously.

In one embodiment, the delivery device is defined by an elongate tube having five functional zones. In the first zone, the orientation zone, a majority of sperm (1) exiting a sample injection tube (110), (preferably having a bevelled injection tip to minimise the effects to the laminar flow of the sheath fluid entering the nozzle via entry points (18)), are oriented into the desired position for analysis at testing zone (10). The unique internal geometry of the nozzle combined with the laminar flow of the sheath fluid create special hydrodynamic forces which provide a stream of sperm, a significant proportion of which are at the correct orientation for testing. The maintenance of a sperm's orientation is achieved via a substantially rectangular cross-sectional tube (5), which is contiguous with a nozzle (8). Downstream of the orientation maintenance zone is a second zone, the testing zone (10). After analysis unwanted sperm are immobilised or eliminated at a third zone. The fourth zone is a deceleration pre-collection area (‘3-3’) before selected viable sperm of a desired sex are collected in a fifth zone, the collection zone. The collected cells are maintained in a suitable environment (4,41) for post-selection use.

The testing zone (10) is substantially a cavity, tube or aperture that is of a size and shape sufficient to accommodate and maintain the orientation of individual cells and which allows for testing, analysis and consequent selection of those cells meeting desired criteria. In one embodiment the short axis of the rectangular testing zone when looked at in cross section is approximately 32 μm, the long axis 70 μm.

In particular, the rectangular cross-sectioned tube (5) maintains the orientation created by the nozzle (8) of delicate cells and allows the cells to pass in single file into the testing zone (10) through a first light source. The first light source is preferably derived from a laser. Sperm cells stained with an appropriate fluorochrome, such as SYBR I, SYBR II, SYBR gold bisbenzimide H 33342, are excited, fluoresce and the magnitude of fluorescence is measured. SYBR II, for example has a peak excitation at 488 nm and peak emission at 525 nm.

Simultaneous to the above, the individual sperm cells are exposed to a second and different light source, such as light derived from an optical phase contrast or Dark field system. This light source is projected orthogonal to the first light source. The sperm being tested emits light. The light is captured, amplified and analysed by a multi-channel analyser or appropriate computer analysis tool.

The fluorescence emitted as the sperm passes through the first light source is used to identify whether the sperm carries an X or Y chromosome. The non-fluorescent light refracted by the sperm cell from the second different light source provides for an improved determination of its orientation.

### EXAMPLE 3

Having regard to FIGS. 3-6 the inventor uses the novel hydrodynamic radial orienting nozzle (8) described in Example 2 to radially orient individual sperm into a rectangular cross-sectioned capillary tube located at the nozzle “exit” (‘A-A’). The exit of the orientation nozzle (8) is contiguous with the testing zone (10). This enables spermatozoa emitted from the sample injection tube (110) to develop the ideal radial and focal plane orientation whilst passing through the nozzle as required for optical analysis. As a consequence, a much higher proportion of individual sperm entering the testing zone (10) will be correctly aligned to the fluorescence objective (11) to facilitate the identification of the chromosome complement of individual spermatozoa within the testing zone (10). Downstream of the testing zone a high-speed laser (20) permanently immobilises or destroys spermatozoa of indeterminate sex i.e. not correctly oriented and also spermatozoa of the non-desired chromosome complement.

Upon completion of processing, the spermatozoa are gently decelerated through a gently tapered deceleration zone or pre-collection area (‘3-3’) into a collection vessel (not shown).

This unique pre-collection/deceleration zone is a gently flared continuation of the capillary tube. It has been observed that the degree of divergence and length of flare directly influence deceleration speed. The pre-collection zone in one embodiment takes the form of a “P” trap (4) and is situated at the end of the deceleration zone. The “P” trap (4) is pre-filled with spermatozoa diluent to the level shown (41) prior to commencing processing to stop jetting from the analysis/processing zone.

For In-Vitro fertilisation purposes the immotile/dead spermatozoa may be removed through percoll density gradient centrifugation or swim-up techniques as is pro form a for IVF. The skilled reader will understand that any non-viable, immotile or dead spermatozoa are of no concern in In-Vivo insemination applications.

Examples 1 to 3 illustrate a key difference between the existing art and the present invention, namely that the use of a phase contrast or Dark field optical system, or similar, is used to determine orientation of individual spermatozoons (90 degree detector) with respect to the DNA detector (0 degree detector). This provides for surprising system efficiency gains, and also provides for higher processing speeds and increased accuracy of analysis, through non-overloading of the Photo Multiplier Tube (PMT).

### EXAMPLE 4

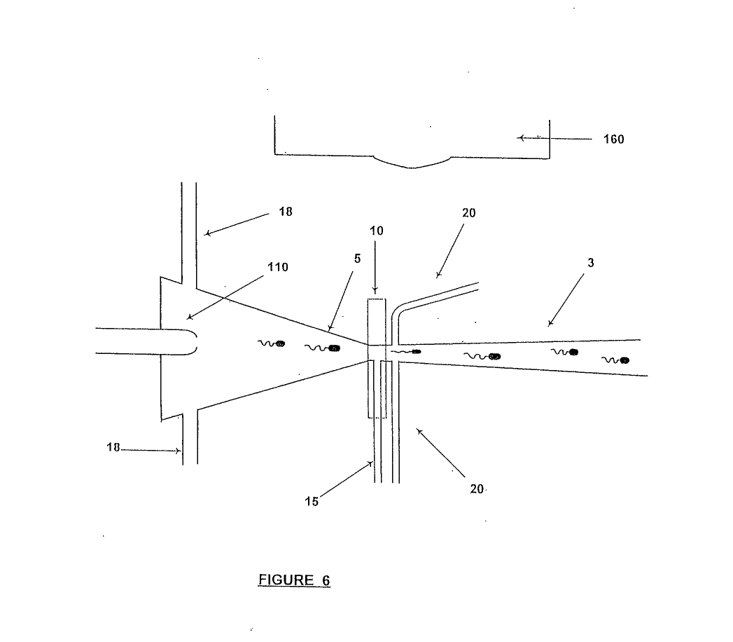

A significantly high proportion of sperm that pass from the sample injection tube (110) and through the hydrodynamic orientation nozzle (8) are correctly aligned before entering the testing zone (10) as described above. On entering the testing zone individual sperm are simultaneously analysed for orientation using phase contrast or Dark field optics (16, 22, 22A) at 90 degrees and for DNA content utilising a fluorescence detector at 0 degrees (11) as shown in FIG. 6. Data is collected and processed via a CPU/analyser (19) and sperm of a desired sex selected utilising an immobilising external triggered Q-switched laser (20), preferably emitting at the 2.69 μm wavelength, although other wavelengths may be used or, an ablative external triggered Q-switched laser utilising other wavelengths may be used.

Needless to say, the selection/immobilisation stage takes place downstream of the testing zone and before entering the deceleration/pre-collection area. It has been found that the 2.69 um laser system is well suited to the sperm sexing method of the invention as it delivers the required power, penetration and absorption characteristics.

The Specifications relevant to the above immobilising Laser (although some specifications may be modified within overall operating requirements) are:
Wavelength 2,690 nm (or 2,620 nm providing for higher penetration but lower absorption, calculations for this wavelength have not been made)
Solid State. Chromium, Thulium, Erbium doped YAG crystal (CTE:YAG) (Although diode lasers may be used provided sufficient power can be generated at required repetition rates and pulse duration levels.
External triggered Q switched
Pulse duration approximately 500 ns
Repetition rate—variable up to 300 kHz
   Split beam, pulse delivery through two dehydrated (low OH) silica optical fibres or hollow rectangular low OH glass fibres positioned directly opposite each other. Surface measurements of internal core or hollow centre delivery fibres at sample interface=70×10 um rectangle with rounded ends, shaped from a flattened optical fibre of 30 um (inner core) diameter, or the electromagnetic energy may be delivered by two essentially rectangular cross-sectioned hollow cored, low OH glass fibres, the hollow section being approximately 70 μm×32 μm.
   External triggered Q switched Laser will revert to very low power CW Alignment Mode between pulses to maintain the correct internal thermal condition of the resonator.

The reader will be aware that only those cells oriented correctly can be used to predict with accuracy the DNA content and therefore the sex characteristics of the sperm.

All of the features disclosed in this specification (including any accompanying claims, abstract and drawings), and/or all of the steps of any method or process so disclosed, may be combined in any combination, except combinations where at least some of such features and/or steps are mutually exclusive.

Alternative features serving the same, equivalent or similar purpose may replace each feature disclosed in this specification (including any accompanying claims, abstract and drawings), unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

### Advantages

The present invention has one or more of the following advantages:
comparatively inexpensive
allows for impressive sample flow rates
provides increased purity of collected sample
improved viability of selected samples
provides increased efficiencies
easier mechanical operation
improved reliability
increased sample orientation dependability

### Variations

Some preferred aspects of the invention have been described and illustrated by way of example, but it will be appreciated that other variations of and modifications to the invention can take place without departing therefrom.

For example, it is envisaged that although the specification is predominantly directed to the selection of X and Y chromosome-bearing sperm cells the possibility of selecting red or white blood cells from a blood sample or, gram negative bacteria from a suitably prepared sample is also contemplated.

The use of such a method to isolate and select for viruses of interest is also an option.

The skilled reader will also instantly realise that the use of filters to exclude light energy of unwanted wavelength might also vary depending on the sample under investigation. Similarly, although the use of an optical/hollow fibre arrangement at a wavelength of 2.69 μm is preferred for the immobilising or ablative laser referred to in the Examples, less suitable but perfectly adequate wavelengths can be delivered through air. In fact, some potential wavelengths are not suited to fibre delivery. Correspondingly, although fluorescence can be delivered through air, it is preferred that the fluorescence excitation wavelength is delivered via fibre optics,

It will also be understood that any reference to a cell will also be directed to parts of a cell and in particular to components of a cell such as nuclear DNA, mitochondrial DNA, RNA, or to organisms or viruses that have invaded or are not normally found within or associated with said cells or parts of said cells.

This document describes the use of the invention with respect to selecting sperm having a desired sex derived from agriculturally important animals, but a skilled reader will instantly realise that above described methods and apparatus will have application in selecting sperm of a desired sex for all placental mammals.

Throughout the description and claims of this specification the word “comprise” and variations of that word, such as “comprises” and “comprising”, are not intended to exclude other additives, components, integers or steps.

### REFERENCES

1. M. Montag, K. Rink, G. Delacretaz & H. van der Ven, 2,000. Laser-induced immobilisation and plasma membrane permeabilization in human sperm. Human Reproduction, Vol 15, No. 4, 846-852.
2. V. Kachell, et al. 1977. Uniform Lateral Orientation caused by Flow Forces, of Flat Particles in Flow through Systems, Journal of Histochemistry and Cytochemistry, Vol. 25, No. 7, pp. 774-780.
3. XY, Inc. PCT Patent Application 15 Nov. 2001 No. WO 01/85913 A2
4. XY Inc. US patent 12 Aug. 2003, U.S. Pat. No. 6,604,435 B2
5. G. M. Hale and M. R. Querry, Optical constants of water in the 200 nm to 200 um wavelength region, Appl. Opt., 12, 555-563, (1973). Web page—http://omic.ogi.edu/spectra/water/data/hale73.dat
6. U.S. Pat. No. 6,263,745, U.S. Pat. No. 6,357,307 & U.S. Pat. No. 5,985,216 to nozzle systems.
7. WO 98/34094.
8. L. A. Johnson and D Pinkel, “Modification of a Laser-based Flow Cytometer for High Resolution DNA Analysis of Mammalian Spermatozoa”, Cytometry 7:268-273 (1986).

1.-38. (canceled)

39. A first method of determining the orientation of an aspherical cell in a process wherein said orientation is used in a second method to select a desired cell or part thereof from a sample of cells due to differences in size, mass, volume or density of the cell or cell part, the method being characterized in that the orientation of a cell is determined by measuring refracted non-fluorescent light provided by a phase contrast or Dark field optical system, said refracted non-fluorescent light having first been passed through one or more band pass filter(s) sufficient to exclude all wavelengths other than that remaining from the phase contrast or Dark field light source.

40. A method according to claim 39 when used in tandem and, preferably simultaneously with, said second method to select desired cells, or parts of cells, wherein said differences in size, mass, volume or density of cells or cell parts are determined by measuring the DNA content of said cells, or parts thereof.

41. A method for determining the orientation of aspherical cells or parts of cells, according to claim 40 which is further used in a method for selecting X chromosome-bearing sperm from Y chromosome-bearing sperm, by measuring the DNA mass of individual sperm cells.

42. A method according to claim 39 wherein the process of selecting desired cells, or part of cells comprises the following steps:
(i) delivering suitably maintained and oriented cells, or part of cells, from a sample of cells of interest into a testing zone,
(ii) exposing said cell sample of interest to a first light source having a first wavelength,
(iii) exposing said cell sample of interest to a second light source having a second different wavelength,
(iv) collecting and measuring light energy emitted at (ii) and (iii) above via suitable detectors,
(v) analyzing the light collected at (iv) to determine whether desired predetermined parameters are met,
(vi) selecting said cells, or part of said cells, which meets said desired parameters,
(vii) collecting the selected cells in a suitable viability maintenance medium, and/or
(viii) immobilizing, or destroying unwanted cells, or parts of cells.

43. A method according to claim 42 wherein, the cells to be selected are either X chromosome-bearing or Y chromosome-bearing sperm cells.

44. A method according to claim 42 wherein, the sperm cells are stained with a suitable DNA specific binding fluorochrome.

45. A method according to claim 44 wherein, the fluorochrome is selected from SYBR green I, SYBR green II, SYBR gold, and Bisbenzimide H33342.

46. A method according to claim 45 wherein, the first light source is an excitation light source adapted to provide fluorescent light preferably, with a peak excitation at from 488 nm-497 nm.

47. A method according to claim 42 wherein, said first light source is used to fluoresce fluorochrome treated cells so as to produce sufficient fluorescence to measure the DNA content of a sperm cell.

48. A method according to claim 47 wherein, said first light source is used to fluoresce fluorochrome treated cells so as to produce sufficient fluorescence to measure the DNA mass of a sperm cell.

49. A method according to claim 44 wherein, said second light source is used to determine the orientation of said sperm cells.

50. A method according to claim 49 wherein, the second light source comprises part of a phase contrast or Dark field optical system and the one or more band pass filters are sufficient to exclude both the excitation and fluorescent wavelengths emitted from said first light source and any unwanted, aberrant light emitted from an immobilising or ablative means.

51. A method according to claim 50 wherein, the cell is simultaneously exposed to said first and second light source.

52. A method according to claim 51 wherein, sperm cells are passed through a sample orientation device adapted to hydrodynamically orient individual sperm cells such that a majority of sperm cells are presented to said first and second detectors in a uniform fashion, said orientation device being adapted to deliver viable individual sperm cells to a testing zone and wherein, said testing zone is a substantially rectangular receiving area adapted to receive and maintain the orientation of individual sperm cells for detection, DNA measurement, and either selection, immobilization or destruction depending on one or more predetermined selection parameters.

53. A method according to claim 52 wherein, the sperm cells to be tested are delivered to a rectangular testing zone at a flow rate above 5,000 cells per second, and preferably above 10,000 sperm cells per second.

54. A method according to claim 53 wherein each individual sperm cell is analyzed to ensure it is correctly aligned during the testing process, correct alignment indicating a true reading, and wherein once the DNA content of each cell has been measured to determine its sex, the cells are rendered incapable of fertilization, or not, depending on whether the individual sperm cell is of the desired chromosome content and maintained in a suitable medium.

55. An apparatus for selecting desired cells, or parts of cells, the apparatus comprising:
(i) a means for passing suitably maintained and oriented cells from a sample of cells, or parts of cells, of interest into a testing zone,
(ii) a means of exposing said cell sample of interest to a first light source having a first wavelength,
(iii) a means of exposing said cell sample of interest to a second different light source having a different wavelength, wherein the second light source uses phase contrast or Dark field optics and one or more band pass filters sufficient to exclude both the excitation and fluorescence wavelengths emitted from said first light source (ii) and any unwanted, aberrant light from an immobilizing or ablative means (vii) to determine cell orientation,
(iv) separate detection means for collecting and, if necessary, amplifying light emitted by said sample at (ii) and (iii),
(v) a means for analyzing the data collected by separate detection means (iv) to determine whether desired predetermined parameters are met,
(vi) a means for collecting, selecting and maintaining desired cells in viable condition meeting said desired predetermined parameters, and/or
(vii) an immobilizing or ablative means for immobilizing or eliminating unwanted cells or parts of cells not meeting said predetermined parameters.

56. An apparatus according to claim 55 wherein, the cells or parts of cells to be tested are whole, viable sperm cells.

57. An apparatus according to claim 56 wherein the sperm cells are delivered to a rectangular testing zone at a flow rate above 5,000 cells per second, and preferably above 10,000 sperm cells per second.

58. An apparatus according to claim 56 wherein, the sperm cells once selected comprise a substantially homogeneously sexed population of viable sperm cells having either an X chromosome-bearing population or a Y chromosome-bearing population at a purity of greater than 95% and preferably a purity of greater than 98%.

59. An apparatus according to claim 55 wherein, said first light source is an excitation light source, preferably with a peak excitation at a wavelength of 488 nm and providing a peak emission at 525 nm, depending on the DNA-binding fluorochrome used.

60. An apparatus according to claim 55 said first light source is used to analyze the DNA content of a cell.

61. An apparatus according to claim 55 wherein, each individual cell is simultaneously exposed to said first and second different light sources.

62. An apparatus according to claim 55 wherein, said means for collecting light emitted from said sample after exposure to said first light source comprises part of a phase contrast or Dark field optical system.

63. An apparatus according to claim 62 wherein, said means for collecting light emitted from said sample after exposure to said second light source is an optical detector able to collect light energy of a non-fluorescent wavelength.

64. An apparatus according to claim 55 wherein, said analysis means is a multi-channel analyzer or computer programmed with suitably developed computer software.

65. An apparatus according to claim 55 wherein, the means for providing said first light source and immobilizing or ablative means is a fiber optic delivery system, preferably comprising hollow cored, low OH glass fibers.

66. A delivery device when used in a method according to claim 39 adapted to sequentially deliver individual whole, viable sperm cells from a sample injection tube via a hydrodynamic radially orienting nozzle to a testing zone, a deceleration or pre-collection zone and a collection means, the delivery device comprising:
an elongated tube comprising a first end portion and a second end portion,
the first end portion comprising a nozzle,
the second end portion comprising a pre-collection or deceleration zone, and wherein,
said first and second end portions are spaced apart either side of a substantially rectangular cross-sectioned testing zone and wherein,
said first end portion comprising said nozzle has a first end and a second end, said first end being adapted to communicate with a sample injection tube to receive said sample and said second end being contiguous with said testing zone, the nozzle being of a size and shape sufficient to maintain a majority of said sperm cells in a laminar flow at a desired hydrodynamic radial orientation, and
said second end portion comprising a pre-collection or deceleration zone is configured to convey sperm cells to a collection means such that said cells after exiting the testing zone selected sperm cells are maintained in a viable condition suitable for use in an in-vitro or in-vivo fertilization procedure.

67. A delivery device according to claim 66 wherein, the pre-collection or deceleration zone is flared outwards from the substantially rectangular cross-sectioned testing zone.

68. A delivery device according to claim 66 wherein, the pre-collection zone comprises a series of grids or groynes sufficient to decelerate individual sperm as they proceed from said testing zone to said collection means after testing.

69. A delivery device according to claim 66 wherein, in use, as the cells pass from the injection tube and into the delivery device the orientation nozzle maintains the correct orientation of a majority of the individual cells into a position which allows for each individual cell to pass through a first light source having a first wavelength and light emitted by said cell to be detected and analyzed for DNA mass, and which simultaneously allows for said cell to pass through a second different light source having a second different wavelength to be detected and analyzed for correct orientation.

70. A method of selecting a desired viable sperm cell suitable for in-vivo or in-vitro fertilization, the method having the following steps:
(i) staining intact, viable sperm collected from a male mammal with a suitable DNA specific binding fluorescent dye, such that the DNA takes up the fluorescent dye uniformly,
(ii) maintaining the stained sperm in an suitable maintenance medium sufficient to maintain the sperm and/or contained DNA within the cell in a viable condition,
(iii) passing the maintenance medium containing the sperm before a suitable excitation light source causing the stained DNA to fluoresce,
(iv) passing the maintenance medium containing the sperm through both a means for measuring the fluorescence of the stained DNA and a means for detecting the orientation of the sperm, wherein said sperm orientation detection means uses phase contrast or Dark field optics and one or more band pass filters sufficient to exclude both the excitation and fluorescence wavelengths emitted from said suitable excitation light source at step (iii),
(v) collecting light energy emitted by said sperm cell, converting the light energy into electrical signals and analyzing the electrical signals via a multi-channel analyzer or suitably programmed CPU,
(vi) selecting those sperm cells meeting desired predetermined criteria, and
(vii) either immobilizing or destroying cells, which fail to meet, desired predetermined criteria.

71. A method according to claim 70 for producing viable sperm for use in an in-vitro fertilization procedure.

72. A method according to claim 70 for producing viable sperm for use in an in-vivo fertilization procedure.

73. A viable sperm cell, the product of a method according to claim 70.
